(19) United States
(12) Patent Application Publication
(10) Pub. No.: US 2008/0319300 A1
(43) Pub. Date: Dec. 25, 2008

(54) Medical Imaging System

(75) Inventor: Jean-Paul Do-Huu, Cernay La Ville (FR)

Correspondence Address: YOUNG & THOMPSON, 209 Madison Street, Suite 500, ALEXANDRIA, VA, 22314, US

(73) Assignee: DESIGNERS DEVELOPERS DISTRIBUTORS ASSOCIATES (D3A) MEDICAL SYSTEMS, ORLEANS (FR)

(21) Appl. No.: 12/159,855

(22) PCT Filed: Jan. 5, 2007
(86) PCT No.: PCT/FR2007/000011

§ 371 Date: Jul. 2, 2008

(30) Foreign Application Priority Data
Jan. 6, 2006 (FR) 0600129

(51) Int. Cl. A61B 5/00; A61B 6/00

(52) U.S. Cl. 600/407; 378/4

(57) ABSTRACT

Medical imaging system, comprising a movable part (10) composed of a work surface (11) and of a bracket (12) supporting an arm (13) that is equipped with a radiation emitter (14) and a radiation receiver (15), said movable part (10) additionally comprising: means (16) allowing said arm (13) to slide vertically along the bracket (12), and means of rotation (41 to 48) for turning said bracket (12) about a vertical axis (12a). The system can comprise a fixed part (50) with at least one support (51) that can receive part of a patient's body and allows an imaging incidence to be reproduced.

The present invention relates to a medical imaging system. More precisely it relates to a mobile medical imaging system using X-rays.

The field of the invention is the medical field, The invention relates more particularly to the field of high precision medical imaging, by the use of X-rays, of an extremity of the body of a patient such as a wrist, an ankle, etc., or a patient's articulation joint such as a knee, an elbow, etc.,

Currently, a plurality of mobile medical imaging systems and devices exist. Among these mobile systems and devices there can be mentioned the instruments dedicated to mammograms or to dental imaging, so-called “mobile surgical” devices and systems dedicated to medical imaging applications during surgical operations, and so-called “mobile radiological” devices and systems, used for carrying out medical imaging at the patient's bedside.

However, these systems and devices have respective drawbacks. The instruments dedicated to mammograms or to dental imaging cannot be used for a medical imaging of just any part of the body of a patient, and are not autonomous and do not allow coverage of all the operations from taking medical images to making diagnosis tools available. Moreover, the instruments for mammograms or dental imaging do not make it possible to cover certain views for taking an image whilst making it possible to reproduce these views.

The “mobile surgical” systems have problems of space requirements as they are often composed of several assemblies. Moreover, they are impractical to handle, and often lack precision in the reproduction of the views for taking an image.

Similarly, the “mobile radiological” systems are not equipped either with means making it possible to cover all the operations from taking medical images to making diagnosis tools available. For example, they do not comprise the means for processing and displaying a medical image making it possible to perform a diagnosis. The “mobile radiological” systems moreover suffer from a lack of imaging precision.

An objective of the invention is to overcome the drawbacks mentioned above.

Another objective of the invention is to propose a mobile medical imaging system making it possible to cover a very large number of imaging views and to ensure the reproducibility of these views.

An objective of the invention is also to propose a complete and autonomous medical imaging system making it possible to carry out all the operations from taking a medical image to making diagnosis tools available.

The invention also aims to propose a medical imaging system making it possible to take high-resolution medical images.

Another objective of the invention is to propose a more efficient and simpler medical imaging system than the medical imaging systems and devices of the state of the art.

Finally, another objective of the invention is to propose a medical imaging system which is easy to manufacture and is inexpensive.

The invention proposes to overcome the drawbacks mentioned above by a medical imaging system comprising a mobile part composed of a work surface, a pillar stand supporting an arm equipped with an radiation emitter and a radiation receiver, said mobile part comprising moreover.
means for allowing said arm to slide vertically along the pillar stand; and
means of rotation of said pillar stand about a vertical axis;

Advantageously, the system according to the invention comprises a mobile part which can be moved. The movements of this mobile part can be carried out using motorized means which can be arranged so that it is possible to move the mobile part of the system according to the invention without expending significant physical effort. The movements of the mobile part can also be carried out by means of wheels or similar means equipping the mobile part.

The system according to the invention is very practical as it comprises a mobile part which can be moved. As a result, it is possible by using the system according to the invention to move the means of medical imaging up to the patient and not the other way round. This makes it possible to take medical images of a patient who cannot be moved for some reason or other. The system comprising an arm equipped with an emitter and a receiver makes it possible to take medical images with a high imaging precision and allows a plurality of imaging views to be reproduced.

Advantageously, the system according to the invention can be suitable for a plurality of imaging positions. In fact, the freedom of rotation of the pillar stand supporting the wave emitter and receiver makes a plurality of imaging positions possible, as in this way it is no longer necessary to move the whole medical imaging system to take two images in two different directions: all that is required is to turn the pillar stand supporting the wave emitter and receiver.

Due to its composition, the system according to the invention is advantageously simple to produce, implement and use. Moreover, the elements composing the system according to the invention are themselves very simple and easy to produce. Furthermore, the system according to the invention can be manufactured with low weight materials, which by reducing the weight of the system will enhance its practical aspect Moreover, the system according to the invention is not very expensive.

In an advantageous version of the invention, the system according to the invention can comprise means of rotating the arm about a horizontal axis. These means of rotation can be motorized, and thus allow fully automatic rotation of the arm, with little physical effort. In this way the system can be provided with free rotation of the pillar stand about a vertical axis and of the arm about a horizontal axis, the arm being capable of sliding along the pillar stand. This combination of rotations makes it possible for the emitter and receiver assembly to be positioned within a very large area composed of a multitude of positions allowing, for example, a medical image to be taken of a joint and/or a body extremity of a patient.

The systems according to the invention can comprise means making it possible for at least one element composing the system to be balanced, so that it can be moved more easily and remain in a configuration chosen by the user.

Advantageously, the arm equipped with an emitter and a receiver can be arranged to frame, horizontally and close to the ground, a support for one part of the body of a patient. In fact, the freedom in rotation about a vertical axis of the pillar stand and optionally about a horizontal axis of the arm and the freedom of the arm to slide along the pillar stand, make it possible for the arm to be positioned horizontally and close to the ground and thus able to frame a support serving to support a limb of a patient of whom it is desired to take medical images.

Advantageously, the system according to the invention can comprise means for stabilizing the pillar stand and/or the arm in any position. In fact, the arm is free in translation along the pillar stand, which itself is free to rotate about a vertical axis. Moreover, the arm can advantageously be free in rotation about a horizontal axis. The system according to the invention can comprise means of stabilization of at least one element composing the system according to the invention. These means of stabilization can be brakes or “locking screw” type means, or any other equivalent means.

Advantageously, the work surface can comprise a cavity in which the receiver can be fitted so as to be fixed vertically onto said surface. This cavity makes it possible to adjust the receiver on the work surface so that the receiver rests on the work surface. Thanks to this cavity, it is very practical for the user to place the arm equipped with the emitter and the receiver in a vertical position above the work surface. This cavity makes it possible to guide the user in positioning the receiver on the work surface. Moreover, this cavity allows the receiver, when it is fitted in the cavity, to be at the same height as the work surface. It therefore ensures the continuity of the work surface.

In an advantageous version of the invention, the receiver can be arranged so that it is used as a support for at least one part of the body of a patient. In fact, the arm carrying the receiver can be fixed vertically above the work surface. In this position, the receiver can be positioned vertically on the work surface or optionally be inserted into a cavity present on the work surface. In this configuration, the receiver becomes a part of the work surface and can be used as a work surface. It can then be used as a support for a limb or a part of the body of a patient of whom the user desires to take a medical image.

It is very advantageous for the work surface to comprise a cavity in which the receiver can be fitted, as the receiver can be fixed on the work surface so that the upper faces of the receiver and the work surface are in the same plane. Thus, the continuity of the work surface is maintained and the receiver rests on the work surface in a way that supports the weight of a limb or a part of the body of a patient.

Advantageously, the system according to the invention can be used for a medical imaging of an ankle and/or a wrist. In fact, the system according to the invention can be positioned so as to take a medical image of an ankle or a wrist, whether in a vertical position of the arm carrying the wave emitter/receiver assembly, or in a position of this arm which is horizontal and close to the ground. The ankle or the wrist can be positioned so as to be placed over the wave receiver. They can also be placed on a support which will then be framed by the wave emitter and wave receiver assembly. The ankle or the wrist can also be brought up against the receiver according to a view chosen by the user.

More advantageously, the system according to the invention can be used for a medical imaging of a joint of the body of a patient. In fact, the system according to the invention can be used for taking a medical image of a knee, an elbow, a hip, etc, of a patient. It can also be used to take a medical image of an extremity of the body of a patient, Advantageously the system according to the invention can comprise means for protecting at least one part of the body of a patient from the radiation emitted by the emitter and/or a collision with at least one element of the system. In fact, since the radiation emitted by medical imaging systems is injurious to the health of an individual, the system according to the invention can comprise means for protecting a sensitive part of the body of the patient from these waves. Similarly, at least one element of the system can collide with at least one part of the body of a patient, during the positioning of at least one element of the system. The system according to the invention comprises means for protecting the patient against these risks of collision.

Advantageously, at least one element of the system can comprise means of contact detection. For example, the receiver can comprise sensors for detecting a contact, allowing the receiver on the one hand, to be positioned more precisely and on the other hand, to avoid collisions with another element. These sensors can, for example, be located on the perimeter of the receiver.

In an advantageous version of the invention, the system according to the invention can comprise an adjustable screen for displaying a medical image. This adjustable screen can be an electronic screen allowing the images to be displayed in a computerized form.

According to an advantageous feature of the invention, the system according to the invention can comprise computing means for processing a medical image. These computing means can comprise a computer equipped with suitable computing means allowing the processing of images. Moreover, taking the medical images can be carried out in a computerized manner. In fact, the wave receiver can be provided with digital wave sensors. During imaging, these wave sensors can send data to computing means using suitable interfaces. This data can then be processed using software to display the image on an adjustable screen, for example. The means of taking and processing an image can comprise multiple functions; zoom, taking images in bursts, etc.

The system according to the invention can also comprise means for printing an image such as at least one colour printer or otherwise.

In an advantageous version of the invention, the receiver comprises at least one digital sensor making it possible to visualise an element of a size less than or equal to 200 μm. More advantageously, the receiver can comprise a digital sensor making it possible to visualise elements of a size less than or equal to 100 μm.

The digital sensor can be a digital sensor with high spatial resolution, having a resolution of at least 5 line pairs per mm and more advantageously at least 8 line pairs per mm.

In an advantageous version of the invention, the radiation used for taking medical images is X-rays. Thus, the system can comprise an X-ray emitter and an X-ray receiver, provided with X-ray sensors.

The system according to the invention can comprise motorization means, making it possible to position at least one element of the system with little physical effort. The system can also comprise means for controlling these motorization means.

The system according to the invention can also comprise battery means. Thus, it will be possible with the system according to the invention to take medical images of a part of the body of a patient at a location where there is no access to a power source, or outdoors.

Advantageously, the system according to the invention can comprise a fixed part comprising at least one support capable of receiving a part of the body of a patient and/or capable of reproducing an imaging view. The support can be substantially V-shaped, capable of receiving for example a foot, for taking a medical image of an ankle. It can be of any other shape suitable for receiving a part or a limb of the body of a patient. The fixed part of the system can advantageously comprise means for supporting the patient and means, such as for example a chair or a bed, allowing the patient to be positioned.

In an advantageous position, the arm can be vertical, the radiation emitter being at the top of the arm, and the radiation receiver can rest on the work surface. Thus medical images of a limb or a part of the body of a patient can be taken, by placing this limb on the receiver which itself is placed on the work surface.

In an advantageous position, the arm can be vertical, the radiation emitter and the receiver being opposite the work surface. Thus, a medical image of a limb of a patient can be taken without using the work surface. This is particularly advantageous when it is not possible to use the work surface, or when the patient is placed on another appliance such as a bed or a stretcher. For example, if a patient is lying on a bed and cannot be moved, the system according to the invention can be brought close to the bed and the receiver placed directly on the patient's bed and thus take a medical image of one of his limbs.

Finally, in another advantageous position, the arm can be horizontal, the radiation emitter and receiver being opposite the work surface. This position is particularly useful for taking an image of a part of the body of a patient situated close to the ground. Thus, the wave receiver and emitter can be positioned horizontally and close to the ground so as to frame the limb of the patient of whom it is desired to take the images. In this configuration it is possible, for example, to take medical images of an ankle of a patient seated on a chair. The foot of the patient can advantageously be placed on a support close to the ground.

Advantageously, the system will be adapted to the position of the patient's limb and not the other way round.

The system according to the invention is therefore complete and autonomous. It can comprise means making it possible to carry out all the operations ranging from taking a medical image to making diagnosis tools available.

Other advantages and characteristics of the invention will become apparent on examination of the detailed description of an embodiment which is in no way limitative, and the attached diagrams, in which:

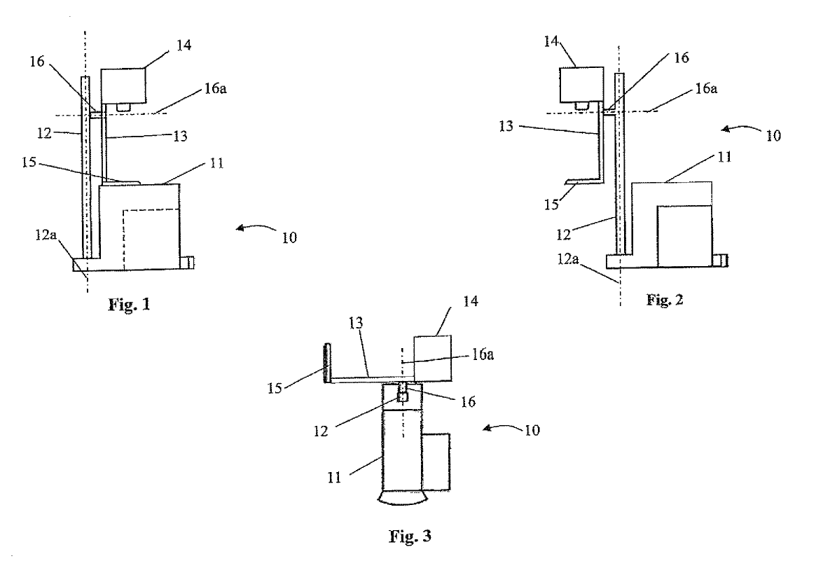

FIGS. 1 to 2 are simplified illustrations of a mobile part of a medical imaging system according to the invention, in side view in two different configurations;

FIG. 3 is a simplified illustration of a mobile part of a medical imaging system according to the invention, in top view;

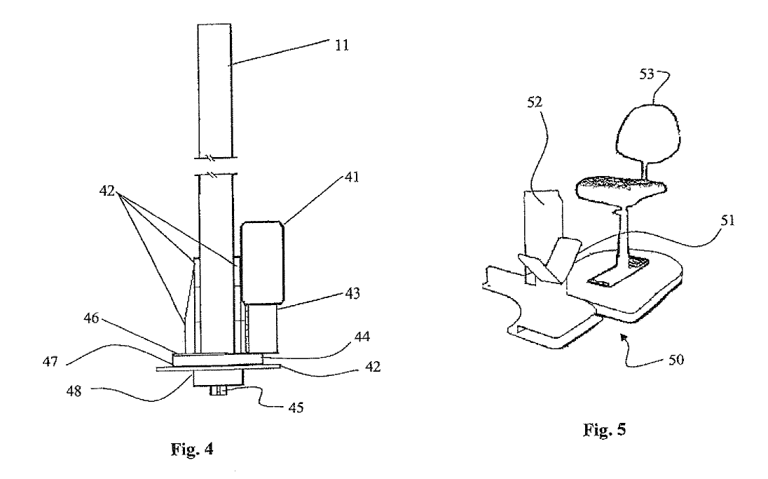

FIG. 4 is a diagrammatic representation of a pillar stand used by a medical imaging system according to the invention;

FIG. 5 shows a mobile part of a medical imaging system according to the invention;

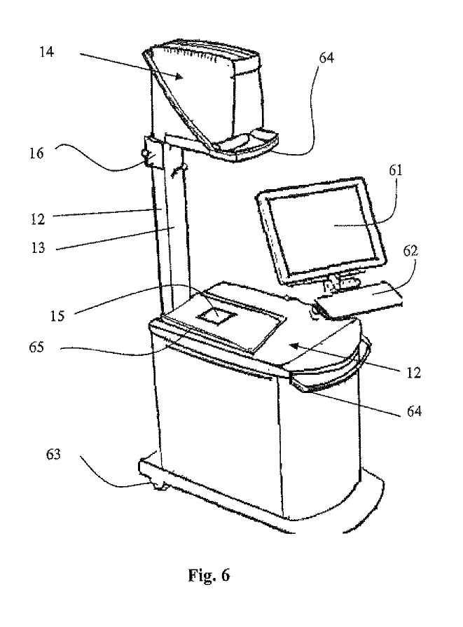

FIG. 6 shows a mobile part of a medical imaging system according to the invention; and

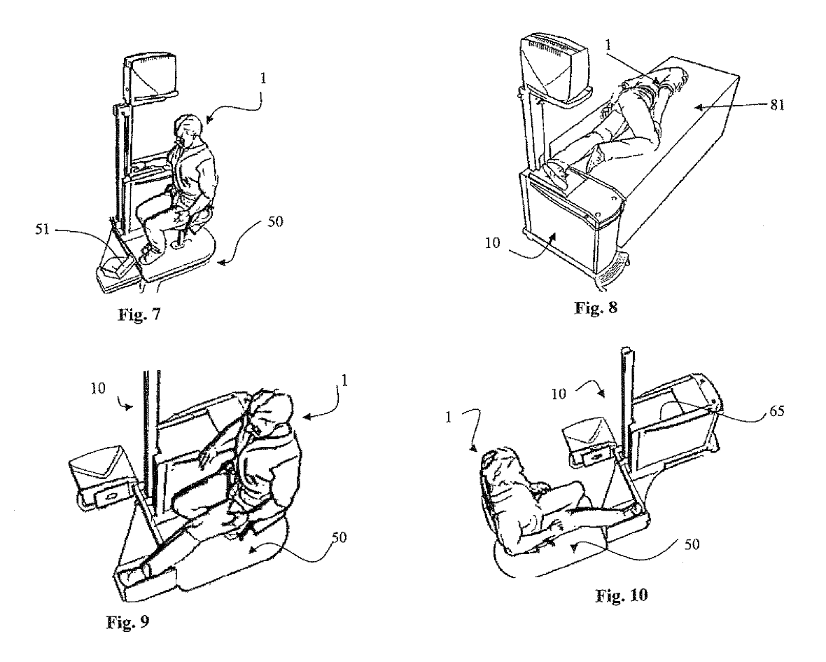

FIGS. 7 to 10 show several positions of a mobile part of a medical imaging system according to the invention.

With reference to FIGS. 1 to 3, the mobile part of a medical imaging system comprises a work surface 11, a pillar stand 12 supporting an arm 13 equipped with an X-ray emitter 14 and an X-ray receiver 15. The system comprises moreover translation means 16 to move the arm 13 along the pillar stand 12.

FIG. 1 shows the mobile part 10 of the system, in profile, in a configuration where the arm 13 is positioned vertically. The X-ray receiver 15 rests on the work surface 11. In FIG. 2 the mobile part of the system is shown in profile in a configuration where the arm 13 equipped with the receiver 15 and the emitter 14 is positioned vertically opposite the work surface 11. FIG. 3 shows a top view of the mobile part in a configuration where the arm 13 is opposite the work surface 11, in a horizontal position. In order to change from the configuration shown in FIG. 2 to the configuration shown in FIG. 3, the arm 13, using the means of rotation and translation 16, performs on the one hand a rotation about a horizontal axis passing through the means of rotation 16, on the other hand a translation along the pillar stand 12. The order of these movements is not important.

To change from the configuration shown in FIG. 1 to the configuration shown in FIG. 2, the pillar stand 12 supporting the arm 13, performs a rotation about a vertical axis passing through the pillar stand. FIG. 4 shows diagrammatically the means making it possible for this pillar stand to perform this rotation. In this figure can be seen the motorization means 41 can be seen which are coupled to gear reduction means 43 to achieve a higher precision of positioning the pillar stand 11 during its rotation, The rotation is carried out using, in particular, a turntable 44, a ratchet wheel 46, slewing ring 47 and a hub 48 welded onto a frame. The assembly shown in FIG. 4 performs a rotation around a rotation shaft 45 and is assembled and reinforced by means of welded sheets 42.

FIG. 6 shows a mobile part of a medical imaging system according to the invention. In this figure there can be seen a work surface 11, a pillar stand 12 supporting an arm 13 equipped with an X-ray emitter 14 and an X-ray receiver 15, means 16 allowing the arm 13, on the one hand to slide along the pillar stand 12 and, on the other hand to perform a rotation about a horizontal axis. In this figure there can also be seen a screen 61 for displaying the medical images taken and for monitoring the operations performed, a keyboard 62 allowing information to be input relating to the patient and to the medical images taken, as well as to control the operations performed. This mobile part can be moved using displacement means composed of wheels 63. Moreover, the mobile part 10 as well as the different elements such as the arm 13 and the pillar stand 12 can be moved by means of multiple fitments 64. The receiver 15 can be fitted in a cavity 65 situated on the work surface so as to be fixed on the work surface while respecting its continuity. The patient can then place one of his limbs on the wave receiver, as in this case, the receiver having been fitted on the work surface 11 can support a greater weight.

FIG. 5 shows a fixed part 50 of a medical imaging system according to the invention. This fixed part 50 comprises a support 51 for at least one part of the body of a patient. The support 51 in this example has a shape substantially similar to a V, so that a patient can place a foot there, for images to be taken, for example, of one of his ankles. This support can also be used for taking all kinds of medical images of the lower limbs of a patient. The support 51 is also used to reproduce at least one imaging view. The fixed part 50 of the system can comprise means, such as a chair or a bed, allowing a patient to position himself or to steady himself for a medical image to be taken. The fixed part of the system can also comprise means of protection, such as a plate 52, of at least one part of the body of a patient, an operator or a bystander, against the radiation involved in medical imaging.

FIGS. 7 to 10 show four configurations for medical imaging with the system according to the invention. Thus, in FIG. 7 the patient is seated on a chair 53 fixed onto the fixed part 50 or not, and places his wrist on the receiver 15. The latter is fixed on the work surface 11 using the cavity 65, the arm 13 is in a vertical position. In FIG. 8, the arm is still in a vertical position, thus allowing the receiver 15 to be fixed on the work surface using the cavity 65. The mobile part of the system is placed alongside a bed 81 on which the patient is lying. This configuration makes it possible to take an image of an ankle of the patient without the patient being moved. In fact, the mobile part of the system is moved and adjusted in relation to the position of the patient without disturbing the patient. This embodies a very useful feature of the system according to the invention. The possibility can also be considered that, in a particular application of the system according to the invention, the bed 81 can form part of the system. FIGS. 9 and 10 illustrate two configurations for taking a medical image of an ankle of a patient. The patient, seated on the chair 53, is either on the same side as the mobile part 10 of the system, in FIG. 9, or the opposite side in FIG. 10. In fact, the fixed part 50 and the mobile part 10 of the system are designed to be capable of being combined, whether on the same side or not and in the same direction or not. In FIGS. 9 and 10 the arm 13 equipped with the emitter 14 and the receiver 15 is positioned horizontally and close to the ground. In this position, the emitter 14 and the receiver 15 frame the support 51 on which the patient places his foot.

Whatever the configuration, the system takes up very little space. In fact, in this example, the system has the following dimensions:
Mobile part
   max. height.: 1882 mm;
   max. length.: 1509 mm; and
   max. width.: 621 mm.
Fixed part
   max. length.: 1261 mm; and
   max. width.: 962 mm.

In the configuration with the greatest overall dimensions, the system comprising one mobile part and one fixed part does not exceed the following dimensions:
max. height: 1882 mm;
max. length.: 2422 mm; and
max. width.: 2070 mm.

The mobile part of the system can operate without the fixed part and can be moved in a practical fashion.

The system according to the invention is not limited to the example described above, being capable of taking varying forms, and can be applied to all kinds of medical imaging.

1-19. (canceled)

20. A medical imaging system comprising a mobile part (10) composed of a pillar stand (12) supporting an arm (13) equipped with a radiation emitter (14) and a radiation receiver (15), said mobile part (10) comprising moreover:
means (16) for allowing said arm (13) to slide vertically along the pillar stand (12); and
means of rotation (41 to 48) of said pillar stand (12) about a vertical axis (12a);
said mobile part (10) moreover comprising a work surface (11).

21. A system according to claim 20, moreover comprising means of rotation (16) of the arm (13) about a horizontal axis (16a).

22. A system according to claim 20, wherein the pillar stand (12) is of a fixed size and the arm (13) is at a fixed distance from the pillar stand (12).

23. A system according to claim 20, moreover comprising means for stabilizing the pillar stand (12) and/or the arm (13) and/or the mobile part (10) in a any position.

24. A system according to claim 20, wherein the work surface (11) comprises a cavity (65) in which the receiver (15) can be fitted in order to be fixed on said work surface (11).

25. A system according to claim 24, wherein the receiver (15) is used as a support for at least one part of the body of a patient.

26. A system according to claim 20, wherein the arm (13) equipped with an emitter (14) and a receiver (15) is arranged to frame, horizontally and close to the ground, a support (51) for one part of the body of a patient.

27. A system according to claim 20, characterized in that it comprises moreover means of protection (52) of at least one part of the body of a patient from the radiation emitted by the emitter (14) and/or a collision with at least one element of the system.

28. A system according claim 20, moreover comprising an adjustable screen (61) for displaying a medical image.

29. A system according claim 20, moreover comprising computer means for processing a medical image.

30. A system according to claim 20, wherein the radiation is X-rays.

31. A system according to claim 20, moreover comprising means of motorization (41) making it possible to position at least one element of said system.

32. A system according to claim 20, wherein the receiver (15) comprises at least one digital sensor making it possible to visualize an element of a size less than or equal to 200 μm.

33. A system according to claim 20, moreover comprising a fixed part (50) comprising at least one support (51) which can receive a part of the body of a patient and/or means of reproducing an imaging view.

34. A system according to claim 20, wherein in one position the arm (13) is vertical, the radiation emitter (14) is at the top of the arm (13), and the radiation receiver (15) rests on the work surface (11).

35. A system according to claim 20, wherein in one position the arm (13) is vertical, the radiation emitter (14) and the radiation receiver (15) being opposite the work surface in relation to the pillar stand (12).

36. A system according to claim 20, wherein in one position the arm (13) is horizontal, the radiation emitter (14) and the radiation receiver (15) being opposite the work surface in relation to the pillar stand (12).

37. Method of taking a medical imaging of an ankle and/or wrist, which comprises using the system according to claim 20.

38. Method of taking a medical imaging of a joint of the body of a patient, which comprises using the system according to claim 20.
